(19) United States
(12) Patent Application Publication
(10) Pub. No.: US 2026/0027109 A1
(43) Pub. Date: Jan. 29, 2026

(54) ORALLY DISINTEGRATING PHARMACEUTICAL TABLET CONTAINING CARIPRAZINE

(71) Applicant: Richter Gedeon Nyrt., Budapest (HU)

(72) Inventors: Melinda Konta, Budapest (HU); Gergely Szakonyi, Budapest (HU)

(21) Appl. No.: 18/997,890

(22) PCT Filed: Aug. 4, 2023
(86) PCT No.: PCT/IB2023/057889

§ 371 Date: Jan. 23, 2025

(30) Foreign Application Priority Data
Aug. 5, 2022 (HU) P2200312

(51) Int. Cl. A61K 31/496; A61K 9/00; A61K 9/20

(52) U.S. Cl. CPC A61K 31/496; A61K 9/0056; A61K 9/2013; A61K 9/2018; A61K 9/2059

(57) ABSTRACT

An orally disintegrating pharmaceutical tablet comprising: between 0.1 and 21 wt % of cariprazine or its pharmaceutically acceptable salts, between 0.1 and 15 wt % of a disintegrant, between 0.1 and 5 wt % of colloidal silicon dioxide, between 0.1 and 10 wt % of a taste masking agent, between 0.1 and 5 wt % of a lubricant, and up to 100 wt % of a diluent, the percentage being expressed with regard to the total weight of the pharmaceutical tablet. Said composition for use in the treatment and/or prevention of pathological conditions which require the modulation of dopamine receptors, selected from the group of psychoses, drug abuse, cognitive impairment accompanying schizophrenia, mild-to-moderate cognitive deficits, dementia, psychotic states associated with dementia, eating disorders, attention deficit disorders, hyperactivity disorders in children, psychotic depression, bipolar disorder, paranoid and delusional disorders, dyskinetic disorders, anxiety, sexual dysfunction, sleep disorders, emesis, aggression and autism.

### FIELD OF THE INVENTION

The present invention relates to orally disintegrating pharmaceutical tablets comprising cariprazine, or pharmaceutically acceptable salts thereof. The invention also relates to said pharmaceutical tablets for use in the treatment and/or prevention of pathological conditions which require the modulation of dopamine receptors.

### BACKGROUND OF THE INVENTION

Cariprazine is a dopamine D₃ receptor and D₂ receptor partial agonist with high selectivity for the D₃ receptor. WO 2005/012266 A1 discloses cariprazine and its use for the treatment and/or prevention of pathological conditions which require the modulation of dopamine receptors, such as psychoses (e.g. schizophrenia and schizo-affective disorders), drug abuse (e.g. alcohol, cocaine, nicotine and opioids), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, bipolar disorder, paranoid and delusional disorders, dyskinetic disorders (e.g. Parkinson's disease, neuroleptic induced parkinsonism, tardive dyskinesias) anxiety, sexual dysfunction, sleep disorders, emesis, aggression and autism.

The current form of administration of cariprazine is an immediate release (IR) capsule, sold under the brand name Vraylar in the United States of America for the treatment of schizophrenia in adults, acute treatment of manic or mixed episodes associated with bipolar I disorder in adults, and the treatment of depressive episodes associated with bipolar I disorder (bipolar depression) in adults, and in the European Union under the brand name Reagila for the treatment of schizophrenia in adults. The capsules must be administered daily to patients in need thereof, due to the fast release mechanism of the active compound. For the purpose of producing a therapeutic effect in a patient, the total daily dose of cariprazine ranges from about 0.5 mg to about 6 mg. However, inter-individual differences and co-medication may necessitate dose titrating in patients. The longer the treatment, the higher the need for an easier intake dosage regimen, since the effectiveness of long-term therapy is closely related, among other factors, to the patients' compliance. The patients' compliance in peroral administration is even more critical in the paediatric and geriatric population where swallowing pharmaceutical formulations is often very difficult due, among other aspects, to the size and the taste of the single dosage form.

CN 107970217 A discloses an orally disintegrating tablet composition comprising cariprazine hydrochloride, binder, disintegrant, thinner and lubricant. The content of the disintegrant must be between 16 and 48 wt %, based on the total amount of the orally disintegrating tablet. Preferred concentration ranges of the disintegrant lie between 18 wt % and 48 wt %, 20 wt % and 48 wt %, 22 wt % and 48 wt %, 22 wt % and 47 wt %, 24 wt % and 46 wt % and between 25 wt % and 45 wt %. The mechanical properties of the orally disintegrating tablets disclosed in CN 107970217 A, such as the friability, are affected by the high concentration of disintegrant in the tablet.

The taste and tablet texture of an orally disintegrating pharmaceutical formulation comprising cariprazine should be taken into consideration in the clinical practice. It is well known that the taste and texture threshold and perception could be significantly altered under pathological conditions. There are several studies supporting this observation, for example, in a survey it was shown, that participants affected by bipolar disorders experience highly erratic taste senses, i.e., they sense the same type of food differently during the maniac and the depressive period (Altered Sensory Phenomena Experienced in Bipolar Disorder (Am J Psychiatry 174:12, December 2017). In another study the relationship between major depression and changes in smell or taste was investigated. It was concluded that there is a strong association between major depression and olfactory and gustatory dysfunction among adults (Association of Alterations in Smell and Taste with Depression in Older Adults Laryngoscope Investigative Otolaryngology 3: April 2018).

The literature generally defines orally disintegrating tablets (ODTs) as solid dosage forms which disintegrate rapidly within a matter of seconds when placed under the tongue. The officially required disintegrating time for ODTs can be differed seconds to minutes related to their size and formulation (US FDA, 2003, Hirani et al., 2009, Thakur and Narwal, 2012). The ideal orally disintegrating tablet has the following properties: no water requirement for oral administration, good swallowability, quick disintegration in mouth, good bioavailability, pleasant taste, sufficient mechanical strength.

There is therefore a need to provide an orally disintegrating cariprazine formulation that improves patients' acceptance and compliance while maintaining good manufacturability and bioavailability of the product.

### DESCRIPTION OF THE FIGURES

FIGS. 1 and 2 show the results of the of the e-tongue measurements, evaluated by PLS (Partial Least Squares) regression, showing an estimation of the bitterness of each sample compared to quinine solutions.

### SUMMARY OF THE INVENTION

The present invention provides an orally disintegrating pharmaceutical tablet comprising:
between 0.1 and 21 wt % of cariprazine or its pharmaceutically acceptable salts,
between 0.1 and 15 wt % of a disintegrant,
between 0.1 and 5 wt % of colloidal silicon dioxide,
between 0.1 and 10 wt % of a taste masking agent,
between 0.1 and 5 wt % of a lubricant, and
up to 100 wt % of a diluent,
the percentage being expressed with regard to the total weight of the pharmaceutical tablet.

The present invention also provides said orally disintegrating pharmaceutical tablet, for use in the treatment and/or prevention of pathological conditions which require the modulation of dopamine receptors, such as psychoses (e.g. schizophrenia and schizo-affective disorders), drug abuse (e.g. alcohol, cocaine, nicotine and opioids), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, bipolar disorder, paranoid and delusional disorders, dyskinetic disorders (e.g. Parkinson's disease, neuroleptic induced parkinsonism, tardive dyskinesias) anxiety, sexual dysfunction, sleep disorders, emesis, aggression and autism.

The present invention also provides the use of said orally disintegrating pharmaceutical tablet in the manufacture of a medicament for the treatment and/or prevention of pathological conditions which require the modulation of dopamine receptors, such as psychoses (e.g. schizophrenia and schizo-affective disorders), drug abuse (e.g. alcohol, cocaine, nicotine and opioids), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, bipolar disorder, paranoid and delusional disorders, dyskinetic disorders (e.g. Parkinson's disease, neuroleptic induced parkinsonism, tardive dyskinesias) anxiety, sexual dysfunction, sleep disorders, emesis, aggression and autism.

The present invention also provides a method of treating a patient suffering from pathological conditions which require the modulation of dopamine receptors, such as psychoses (e.g. schizophrenia and schizo-affective disorders), drug abuse (e.g. alcohol, cocaine, nicotine and opioids), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, bipolar disorder, paranoid and delusional disorders, dyskinetic disorders (e.g. Parkinson's disease, neuroleptic induced parkinsonism, tardive dyskinesias) anxiety, sexual dysfunction, sleep disorders, emesis, aggression and autism, wherein the method comprises the administration of said orally disintegrating pharmaceutical tablet to a patient in need thereof.

The orally disintegrating pharmaceutical tablet of the present invention improves the compliance in patients with swallowing difficulties, including paediatric and geriatric patients. It can be administered daily in an efficient, cost-effective and convenient way for a lifelong therapy and/or prevention of the pathological conditions mentioned above.

### DETAILED DESCRIPTION OF THE INVENTION

The orally disintegrating pharmaceutical tablet of the present invention comprises a therapeutically effective amount of cariprazine, or its pharmaceutically acceptable salts, a diluent, a disintegrant in an amount between 0.1 and 15 wt %, colloidal silicon dioxide in an amount between 0.1 to 5 wt %, a taste masking agent in an amount between 0.1 to 10 wt %, and a lubricant in an amount between 0.1 and 5 wt % of the total weight of the pharmaceutical tablet.

In general, orally disintegrating tablets may have the following problems: poor mechanical strength of the tablets, improper disintegration, dissolution problems, degradation and inhomogeneity of the active pharmaceutical ingredient. During the formulation experiments of the cariprazine containing orally disintegrating tablets we have encountered several problems. Increasing the amount of cariprazine in the tablet matrix reduced the mechanical and degradation problems but increased the dissolution and manufacturability problems. Facing such a complex effect of the active pharmaceutical ingredient on the final product, it was cumbersome to overcome all the formulation and quality problems for all the desired dose strengths, i.e., for dose strengths between 0.01 to 6 mg. The different problems that arose could only be addressed in a complex manner by careful selection of the excipients. Due to the fact of the dose-selective problems, one particular formulation solution caused improvement in some of the tablet parameters at one dose strength but increased quality problems in other dose strengths.

The directly compressible matrix of the ODTs contains mainly fillers, which only provides a low-level interparticle bonding within the tablet matrix. This allows fast disintegration but causes poor mechanical strength. The addition of a disintegrant to the ODTs further improves the disintegration but makes the tablets even more susceptible to friability and cracking or capping/lamination. Moreover, the disintegrant component also helps the dissolution of cariprazine, especially at the higher dose strengths. On the other hand, the amount of the disintegrant must be limited in order to maintain satisfactory mechanical strength. However, it was observed, that a lower amount of the disintegrant can cause dissolution problems.

The pharmaceutical tablet of the present invention is primarily designed for oral disintegration in the patient's mouth, or it can be administered after dissolving it in a suitable volume of liquid, such as water.

Moreover, the pharmaceutical tablet of the present invention is characterized by low friability, high physical stability at high temperatures and humidity levels (30 and 40° C./75% RH), and it satisfies all measurable quality attributes. The orally disintegrating tablet of the present invention can be stored and used at room temperature for more than two years, and also in climate zones characterized by high temperature and high humidity (30° C./75% RH).

The pharmaceutical tablet of the present invention does not require special manufacturing processes. It can be manufactured by mixing and directly compressing the ingredients, in an easy and cost-effective industrial process. The overall therapy costs, which may incur over the entire life of the patient, can therefore be substantially reduced.

In the context of the present invention, the term “orally disintegrating pharmaceutical tablet” means an uncoated tablet intended to be placed in the mouth where it disperses rapidly before being swallowed. Orally disintegrating pharmaceutical tablets disintegrate in the mouth within 3 minutes, within 2 minutes, within 1 minute or within 30 seconds. Recently, European Pharmacopoeia has used the term of orodispersible tablet for tablets that disperses readily and within 3 minutes in mouth before swallowing. United States Food and Drug Administration (FDA) defined the orally disintegrating tablet as a solid dosage form containing medicinal substance or active ingredient which disintegrates rapidly usually within a matter of seconds when placed upon the tongue.

Unless otherwise indicated herein, the term “pharmaceutically acceptable salts” refers to salts obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

Moreover, several acid salts can be obtained by reaction with inorganic or organic acids, namely acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

The disintegrants used in accordance with the present invention are substances or mixture of substances added to the drug formulation that facilitates the breakup or disintegration of the pharmaceutical tablet into smaller particles so that they can disperse or dissolve in the mouth of the patient more rapidly. Disintegrants can increase the bioavailability of the administered active compound through increasing the disintegration and the dissolution from the solid dosage form. The main function of the disintegrants is to counteract the physical forces developing during tablet compression and/or the effect of tablet binders. There is a subgroup within the family of the disintegrants known as superdisintegrants that are advantageously used in orally disintegrating formulations due to their high water absorbing and swelling capacity. Disintegrants which are suitable in accordance with the present invention are, for example, starches, modified starches, such as sodium carboxymethyl starch (sodium starch glycolate), celluloses, modified celluloses, such as croscarmellose sodium, cross-linked polyvinylpyrrolidone (crospovidone), soy polysaccharide, cross-linked alginic acid, gellan gum, xanthan gum, calcium silicate and ion exchange resins, such as indion 414.

As used herein, “diluents” (also referred to as a “filler” or “dilutant”) is a diluting agent. Diluents act as fillers in pharmaceutical tablets and capsules to increase weight and improve content uniformity. Diluents also provide better tablet properties such as improved cohesion or promote flow. Diluents must be non-toxic, physiologically inert, and physically and chemically stable by themselves as well as in combination with active pharmaceutical ingredients. They are inactive ingredients that are added to tablets and capsules in addition to the active drug.

The diluents used in accordance with the present invention may be any diluent conventionally used in the preparation of orally disintegrating tablets. Suitable diluents include sugars, such as lactose, or lactose derivatives, sugar alcohols, such as mannitol, microcrystalline cellulose, starch, starch derivatives or combinations thereof.

As used herein, “glidant” is a non-toxic, flavor neutral, pharmacologically inactive substance used to optimize the flow properties of tablet granulation or powder materials by decreasing interparticle friction and cohesion. Generally, glidants may inhibit the flow properties above a certain concentration. Suitable glidants include corn starch, silica derivatives (colloidal silicon dioxide), syloid, pyrogenic silica, and hydrated sodium sulfoaluminate.

As used herein, “lubricant” is an agent added to tablet and capsule formulations in a very small quantity (usually between 0.1 and 5 wt %) to improve the powder processing properties of formulations. Suitable lubricants include metallic salts of fatty acids such as magnesium stearate and stearic acid, fatty acid esters, inorganic materials, and polymers.

As used herein, “therapeutically effective amount” of a compound means an amount sufficient to cure, alleviate or partially arrest the clinical manifestations of a given disease and its complications in a therapeutic intervention comprising the administration of said compound. The therapeutically effective amount will vary depending on, inter alia, the disease and its severity, and on the age, weight, physical condition and responsiveness of the patient to be treated.

As used herein, “treatment” and “treating” refers to the management and care of a patient for the purpose of combating a condition, such as a disease or a disorder. The term is intended to include the full spectrum of treatments for a given condition from which the patient is suffering, such as administration of an active compound to alleviate the symptoms or complications, to delay the progression of the disease, disorder or condition, to alleviate or relieve the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications.

Unless otherwise indicated herein, all percentages are weight percentages, based on the total weight of the pharmaceutical tablet.

The present invention also relates to an orally disintegrating pharmaceutical tablet comprising:
between 0.1 and 21 wt % of cariprazine or its pharmaceutically acceptable salts,
between 0.1 and 15 wt % of a disintegrant,
between 0.1 and 5 wt % of colloidal silicon dioxide,
between 0.1 and 10 wt % of a taste masking agent,
between 0.1 and 5 wt % of a lubricant, and
up to 100 wt % of a diluent
the percentage being expressed with regard to the total weight of the pharmaceutical tablet.

In one embodiment cariprazine or its pharmaceutically acceptable salts is present in amount between 0.1 and 15 wt %, between 0.2 and 12 wt %, between 0.25 and 10 wt %, between 1 and 5 wt %, or between 0.39 and 4.67 wt % of the total weight of the pharmaceutical tablet.

In an embodiment, the pharmaceutically acceptable salt can be the hydrochloride salt, the hydrobromide salt or the mesylate salt. In another embodiment, the pharmaceutically acceptable salt is the hydrochloride salt.

In one embodiment cariprazine hydrochloride is present in amount between 0.1 and 15 wt %, between 0.2 and 12 wt %, between 0.25 and 10 wt %, between 1 and 5 wt %, or between 0.39 and 4.67 wt % of the total weight of the pharmaceutical tablet.

In one embodiment of the present invention, the disintegrant is a superdisintegrant. In yet another embodiment, the disintegrant is sodium starch glycolate, crospovidone or croscarmellose sodium.

In another embodiment, the disintegrant is present in an amount between 0.1 and 12 wt %, between 0.2 and 12 wt %, between 0.1 and 10 wt %, between 1 and 10 wt %, between 1 and 5 wt %, or between 3 and 5 wt % of the total weight of the pharmaceutical tablet.

In another embodiment, the pharmaceutical tablet of the present invention has a disintegrating time not exceeding 30 seconds. The disintegration time is determined in accordance with the European Pharmacopoeia (9th Edition, 2.9.1. Disintegration of tablets and capsules Test A).

In an embodiment of the present invention, the diluent comprises mannitol, dextrates, isomalt, sorbitol, and mixtures thereof with starch. In another embodiment, the diluent is a mixture of mannitol and starch. Such a mixture is available, for example, from the company Roquette under the tradename Pearlitol® Flash, which comprises 78.0-82.0 wt % D-mannitol and 15.0-19.0 wt % maize starch.

In yet another embodiment, the diluent is present in an amount between 75 wt % and 85 wt % of the total weight of the pharmaceutical tablet.

According to an embodiment of the present invention the taste masking agent is selected from the group of artificial sweeteners, flavoring agents and organic acidic components.

According to another embodiment, the taste masking agent is present in an amount between 0.1 and 8 wt %, between 0.1 and 5 wt % or between 0.5 and 1.5 wt % of the total weight of the pharmaceutical tablet.

In a further embodiment the taste masking agent is an organic acidic component selected from oxalic acid, maleic acid, succinic acid, citric acid, tartaric acid, malic acid, ascorbic acid, fumaric acid, adipic acid. In another embodiment the pharmaceutical tablet further comprises an anhydride of said organic acids; or an inorganic acid salt including, but not limited to sodium dihydrogen phosphate, disodium dihydrogen pyrophosphate and sodium acid sulphite, moreover, the pharmaceutical tablet may comprise a mixture of said organic acids, and/or anhydrides of said organic acids and/or said inorganic acid salts.

In one embodiment the organic acidic component is selected from oxalic acid, maleic acid, succinic acid, citric acid, tartaric acid, malic acid or combinations thereof. In yet a further embodiment, the organic acidic component is malic acid.

According to another embodiment, the organic acidic component is present in an amount between 0.1 and 10 wt %, between 0.1 and 5 wt %, or between 0.5 and 1.5 wt % of the total weight of the pharmaceutical tablet.

In an embodiment of the invention, the orally disintegrating pharmaceutical tablet of the present invention comprises colloidal silicon dioxide as a glidant, such as, anhydrous colloidal silicon dioxide, hydrated colloidal silicon dioxide, and hydrophobic colloidal silicon dioxide.

In yet a further embodiment, the colloidal silicon dioxide is anhydrous or hydrated colloidal silicon dioxide. In a further embodiment, the colloidal silicon dioxide is hydrated colloidal silicon dioxide. According to another embodiment, the colloidal silicon dioxide is present in an amount between 0.1 wt % and 2 wt %, between 0.1 wt % and 1 wt %, or between 0.1 wt % and 0.5 wt % of the total weight of the pharmaceutical tablet.

In a further embodiment of the present invention, the pharmaceutical tablet comprises a lubricant selected from calcium stearate, glycerol behenate, magnesium stearate, mineral oil, polyethylene glycol, sodium stearyl fumarate, stearic acid, talc, vegetable oil, zinc stearate, or combinations thereof.

In an embodiment of the invention, the lubricant is selected from metal stearates, such as magnesium stearate, stearic acid, sodium stearyl fumarate, talc or combinations thereof.

The pharmaceutical tablet may further comprise one or more additional excipients such as binders, granulating aids, effervescent agents and film formers. These excipients may be used in conventional manner, either alone or in any combination.

In a further embodiment, the pharmaceutical tablet of the present invention comprises:
between 0.1 and 21 wt % of cariprazine or its pharmaceutically acceptable salts,
between 0.1 and 15 wt % of a disintegrant,
between 0.1 and 5 wt % of colloidal silicon dioxide,
between 0.1 and 10 wt % of a taste masking agent,
between 0.1 and 2.5 wt % of a lubricant, and
up to 100 wt % of a diluent,
the percentage being expressed with regard to the total weight of the pharmaceutical tablet.

In a further embodiment, the pharmaceutical tablet of the present invention comprises:
between 0.2 and 12 wt % of cariprazine or its pharmaceutically acceptable salts,
between 0.1 and 10 wt % of a disintegrant,
between 0.1 and 2.5 wt % of colloidal silicon dioxide,
between 0.1 and 5 wt % of a taste masking agent,
between 0.1 and 2.5 wt % of a lubricant, and
up to 100 wt % of a diluent,
the percentage being expressed with regard to the total weight of the pharmaceutical tablet.

In a further embodiment, the pharmaceutical tablet of the present invention comprises:
between 0.25 and 10 wt % of cariprazine or its pharmaceutically acceptable salts,
between 0.1 and 10 wt % of a disintegrant,
between 0.1 and 2 wt % of colloidal silicon dioxide,
between 0.1 and 5 wt % of a taste masking agent,
between 0.1 and 2.5 wt % of a lubricant, and
up to 100 wt % of a diluent,
the percentage being expressed with regard to the total weight of the pharmaceutical tablet.

In a further embodiment, the pharmaceutical tablet of the present invention comprises:
between 0.1 and 21 wt % of cariprazine hydrochloride,
between 0.1 and 15 wt % of a disintegrant selected from the group of sodium starch glycolate, croscarmellose sodium, and crospovidone,
between 0.1 and 5 wt % of colloidal silicon dioxide,
between 0.1 and 10 wt % of a taste masking agent selected from the group of oxalic acid, maleic acid, succinic acid, citric acid, tartaric acid, and malic acid,
between 0.1 and 2.5 wt % of a lubricant selected from the group of sodium stearyl fumarate, and magnesium stearate, and
up to 100 wt % of a mixture of 78.0-82.0 wt % D-mannitol and 15.0-19.0 wt % maize starch,
the percentage being expressed with regard to the total weight of the pharmaceutical tablet.

In a further embodiment, the pharmaceutical tablet of the present invention comprises:
between 0.2 and 12 wt % of cariprazine hydrochloride,
between 0.1 and 10 wt % of a disintegrant selected from the group of sodium starch glycolate, croscarmellose sodium, and crospovidone,
between 0.1 and 2.5 wt % of colloidal silicon dioxide,
between 0.1 and 5 wt % of a taste masking agent selected from the group of oxalic acid, maleic acid, succinic acid, citric acid, tartaric acid, and malic acid,
between 0.1 and 2.5 wt % of a lubricant selected from the group of sodium stearyl fumarate, and magnesium stearate, and
up to 100 wt % of a mixture of 78.0-82.0 wt % D-mannitol and 15.0-19.0 wt % maize starch
the percentage being expressed with regard to the total weight of the pharmaceutical tablet.

In a further embodiment, the pharmaceutical tablet of the present invention comprises:
between 0.25 and 10 wt % of cariprazine hydrochloride,
between 0.1 and 10 wt % of a disintegrant selected from the group of sodium starch glycolate, croscarmellose sodium, and crospovidone,
between 0.1 and 2 wt % of colloidal silicon dioxide,
between 0.1 and 5 wt % of a taste masking agent selected from the group of oxalic acid, maleic acid, succinic acid, citric acid, tartaric acid and malic acid,
between 0.1 and 2.5 wt % of a lubricant selected from the group of sodium stearyl fumarate, and magnesium stearate, and
up to 100 wt % of a mixture of 78.0-82.0 wt % D-mannitol and 15.0-19.0 wt % maize starch
the percentage being expressed with regard to the total weight of the pharmaceutical tablet.

In a further embodiment, the pharmaceutical tablet of the present invention comprises:
between 0.1 and 21 wt % of cariprazine hydrochloride,
between 0.1 and 15 wt % of a disintegrant selected from the group of sodium starch glycolate, croscarmellose sodium, and crospovidone,
between 0.1 and 5 wt % of colloidal silicon dioxide,
between 0.1 and 10 wt % of malic acid,
between 0.1 and 2.5 wt % of sodium stearyl fumarate,
up to 100 wt % of a mixture of 78.0-82.0 wt % D-mannitol and 15.0-19.0 wt % maize starch,
the percentage being expressed with regard to the total weight of the pharmaceutical tablet.

In a further embodiment, the pharmaceutical tablet of the present invention comprises:
between 0.2 and 12 wt % of cariprazine hydrochloride,
between 0.1 and 10 wt % of a disintegrant selected from the group of sodium starch glycolate, croscarmellose sodium, and crospovidone,
between 0.1 and 2.5 wt % of colloidal silicon dioxide,
between 0.1 and 5 wt % of malic acid,
between 0.1 and 2.5 wt % of sodium stearyl fumarate,
up to 100 wt % of a mixture of 78.0-82.0 wt % D-mannitol and 15.0-19.0 wt % maize starch,
the percentage being expressed with regard to the total weight of the pharmaceutical tablet.

In a further embodiment, the pharmaceutical tablet of the present invention comprises:
between 0.25 and 10 wt % of cariprazine hydrochloride,
between 0.1 and 10 wt % of a disintegrant selected from the group of sodium starch glycolate, croscarmellose sodium, and crospovidone,
between 0.1 and 2 wt % of colloidal silicon dioxide,
between 0.1 and 5 wt % of malic acid,
between 0.1 and 2.5 wt % of sodium stearyl fumarate, and
up to 100 wt % of a mixture of 78.0-82.0 wt % D-mannitol and 15.0-19.0 wt % maize starch,
the percentage being expressed with regard to the total weight of the pharmaceutical tablet.

In a further embodiment, the pharmaceutical tablet of the present invention comprises:
between 0.1 and 21 wt % of cariprazine hydrochloride,
between 0.1 and 15 wt % of a disintegrant selected from the group of sodium starch glycolate, croscarmellose sodium, and crospovidone,
between 0.1 and 5 wt % of anhydrous colloidal silicon dioxide or hydrated colloidal silicon dioxide,
between 0.1 and 10 wt % of malic acid,
between 0.1 and 2.5 wt % of sodium stearyl fumarate, and
up to 100 wt % of a mixture of 78.0-82.0 wt % D-mannitol and 15.0-19.0 wt % maize starch,
the percentage being expressed with regard to the total weight of the pharmaceutical tablet.

In a further embodiment, the pharmaceutical tablet of the present invention comprises:
between 0.2 and 12 wt % of cariprazine hydrochloride,
between 0.1 and 10 wt % of a disintegrant selected from the group of sodium starch glycolate, croscarmellose sodium, and crospovidone,
between 0.1 and 2.5 wt % of anhydrous colloidal silicon dioxide or hydrated colloidal silicon dioxide,
between 0.1 and 5 wt % of malic acid,
between 0.1 and 2.5 wt % of sodium stearyl fumarate, and
up to 100 wt % of a mixture of 78.0-82.0 wt % D-mannitol and 15.0-19.0 wt % maize starch,
the percentage being expressed with regard to the total weight of the pharmaceutical tablet.

In a further embodiment, the pharmaceutical tablet of the present invention comprises:
between 0.25 and 10 wt % of cariprazine hydrochloride
between 0.1 and 10 wt % of a disintegrant selected from the group of sodium starch glycolate, croscarmellose sodium, and crospovidone,
between 0.1 and 2 wt % of anhydrous colloidal silicon dioxide or hydrated colloidal silicon dioxide,
between 0.1 and 5 wt % of malic acid,
between 0.1 and 2.5 wt % of sodium stearyl fumarate,
up to 100 wt % of a mixture of 78.0-82.0 wt % D-mannitol and 15.0-19.0 wt % maize starch,
the percentage being expressed with regard to the total weight of the pharmaceutical tablet.

In another embodiment, the pharmaceutical tablet of the present invention comprises:
between 0.39 and 4.67 wt % of cariprazine hydrochloride,
between 1 and 5 wt % of sodium starch glycolate,
between 0.1 and 0.25 wt % of anhydrous colloidal silicon dioxide or hydrated colloidal silicon dioxide,
between 0.5 and 1 wt % of malic acid,
between 0.5 and 1 wt % of sodium stearyl fumarate, and
up to 100 wt % of a mixture of a mixture of 78.0-82.0 wt % D-mannitol and 15.0-19.0 wt % maize starch,
the percentage being expressed with regard to the total weight of the pharmaceutical tablet.

Another aspect of the present invention relates to an orally disintegrating pharmaceutical tablet as described above, for use in the treatment and/or prevention of pathological conditions which require the modulation of dopamine receptors. In one embodiment, such use comprises the treatment of psychoses, drug abuse, cognitive impairment accompanying schizophrenia, mild-to-moderate cognitive deficits, dementia, psychotic states associated with dementia, eating disorders, attention deficit disorders, hyperactivity disorders in children, psychotic depression, bipolar disorder, paranoid and delusional disorders, dyskinetic disorders, anxiety, sexual dysfunction, sleep disorders, emesis, aggression and autism.

Another aspect of the present invention relates to an orally disintegrating pharmaceutical tablet as described above, for use in the treatment and/or prevention of schizophrenia and/or bipolar disorder and/or autism.

The present invention also provides the use of said orally disintegrating pharmaceutical tablet for the manufacture of a medicament for the treatment and/or prevention of pathological conditions which require the modulation of dopamine receptors, such as psychoses (e.g. schizophrenia and schizo-affective disorders), drug abuse (e.g. alcohol, cocaine, nicotine and opioids), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, bipolar disorder, paranoid and delusional disorders, dyskinetic disorders (e.g. Parkinson's disease, neuroleptic induced parkinsonism, tardive dyskinesias) anxiety, sexual dysfunction, sleep disorders, emesis, aggression and autism.

Another aspect of the present invention relates to the use of said orally disintegrating pharmaceutical tablet for the manufacture of a medicament for the treatment and/or prevention of schizophrenia and/or bipolar disorder and/or autism.

The present invention also provides a method of treating a patient suffering from pathological conditions which require the modulation of dopamine receptors, such as psychoses (e.g. schizophrenia and schizo-affective disorders), drug abuse (e.g. alcohol, cocaine, nicotine and opioids), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, bipolar disorder, paranoid and delusional disorders, dyskinetic disorders (e.g. Parkinson's disease, neuroleptic induced parkinsonism, tardive dyskinesias) anxiety, sexual dysfunction, sleep disorders, emesis, aggression and autism, wherein the method comprises the administration of said orally disintegrating pharmaceutical tablet to a patient in need thereof.

Another aspect of the present invention relates to the method of treating a patient suffering from schizophrenia and/or bipolar disorder and/or autism, wherein the method comprises the administration of said orally disintegrating pharmaceutical tablet to a patient in need thereof.

Still another aspect of the present invention relates to a process for the preparation of an orally disintegrating pharmaceutical tablet as described above, comprising the following steps:
a. mixing a therapeutically effective amount of cariprazine, or its pharmaceutically acceptable salts with a diluent and optionally with a disintegrant,
b. compressing the mixture obtained under a. into a solid tablet form.
   The following examples serve to illustrate the invention. However, they should not be understood as restricting the scope of the invention.

### EXAMPLE 1. PREPARATION OF THE ORALLY DISINTEGRATING PHARMACEUTICAL TABLETS

All the tablets of the following examples were made according to the process below:
1. Weighing of the components of the product, then
2. roto-sieving of the components of the product, then
3. container blending of the components of the product, then
4. tableting of the powder blend using a rotary tablet press.

### EXAMPLE 2. TASTE MASKING TEST

The orally disintegrating tablets must have a pleasant taste, since they disintegrate in the mouth of the patient. Therefore, commonly used taste masking agents were tested in the pharmaceutical tablets according to the present invention. We found that the most widespread taste masking agents are less effective in masking the taste of cariprazine.

A multichannel taste sensor system, or e-Tongue was used to evaluate the bitterness suppression in different formulations. The sensors are cross-selective for five basic tastes: sourness, sweetness, bitterness, saltiness, and umami. This cross-selectivity is exhibited by a different response profile for five basic taste components. The sensor can detect the taste in a similar manner to the human gustatory sensation by response patterns of electric potential to taste substances. The sensitivity, reproducibility and durability are superior to those of humans. To different concentrations of quinine—used in the bitterness test—the response patterns of the seven sensors were similar while the electric outputs from each individual sensor differed slightly.

In the first set of experiments, different taste masking agents were investigated in terms of their capability of reducing the bitterness of cariprazine. Malic acid, citric acid, tartaric acid, acesulfame potassium, and orange flavor (in concentrations of 1.4 mg/100 ml; 2.8 mg/100 ml and, 4.2 mg/100 ml) were added to the solution of 1.5 mg/ml cariprazine in purified water and e-tongue measurements were carried out on the samples. Table 1 summarizes the predicted quinine bitterness of the different samples.

TABLE 1
Predicted quinine bitterness of different samples

| | | Predicted |
|---|---|---|
| | | quinine |
| Code | Ingredients | bitterness |
| AC_14 | 1.5 mg/ml cariprazine + 1.4 mg/ml acesulfame | 29.57 |
| | potassium | |
| AC_28 | 1.5 mg/ml cariprazine + 2.8 mg/ml acesulfame | 29.47 |
| | potassium | |
| AC_42 | 1.5 mg/ml cariprazine + 4.2 mg/ml acesulfame | 5.70 |
| | potassium | |
| AS_14 | 1.5 mg/ml cariprazine + 1.4 mg/ml malic acid | −11.99 |
| AS_28 | 1.5 mg/ml cariprazine + 2.8 mg/ml malic acid | −29.34 |
| AS_42 | 1.5 mg/ml cariprazine + 4.2 mg/ml malic acid | −30.20 |
| BS_14 | 1.5 mg/ml cariprazine + 1.4 mg/ml tartaric acid | −25.98 |
| BS_28 | 1.5 mg/ml cariprazine + 2.8 mg/ml tartaric acid | −40.96 |
| BS_42 | 1.5 mg/ml cariprazine + 4.2 mg/ml tartaric acid | −43.17 |
| CS_14 | 1.5 mg/ml cariprazine + 1.4 mg/ml citric acid | −23.71 |
| CS_28 | 1.5 mg/ml cariprazine + 2.8 mg/ml citric acid | −40.25 |
| CS_42 | 1.5 mg/ml cariprazine + 4.2 mg/ml citric acid | −52.21 |
| NA_14 | 1.5 mg/ml cariprazine + 1.4 mg/ml orange flavor | 46.90 |
| NA_28 | 1.5 mg/ml cariprazine + 2.8 mg/ml orange flavor | 22.80 |
| NA_42 | 1.5 mg/ml cariprazine + 4.2 mg/ml orange flavor | 42.43 |
| HA | 1.5 mg/ml cariprazine | 51.72 |
| Q1 | 1.6 μM quinine | |
| Q2 | 8.3 μM quinine | |
| Q3 | 83 μM quinine | |

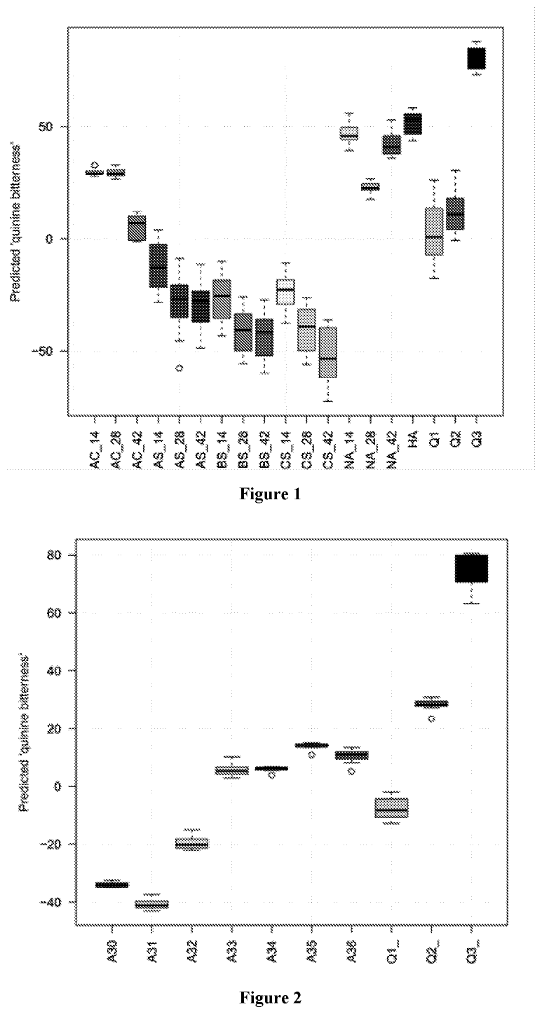

PLS regression was used to compare the taste (bitterness) masking effect of the tested materials, the results are shown in FIG. 1.

Commonly applied taste masking agents, such as orange flavor and the artificial sweetener acesulfame potassium proved to be less effective in reducing the bitterness of cariprazine than organic acidic compounds.

In the second set of experiments, the taste characteristics of a placebo formulation were compared to formulations containing malic acid and cariprazine at different concentrations, as well as to the taste of quinine solutions of different concentrations (Q1—1.6 μM, Q2—8.3 μM. Q3—83 μM). Table 2 shows the composition of the tested samples.

TABLE 2
Composition of the different samples

| | A30 | A31 | A32 | A33 | A34 | A35 | A36 |
|---|---|---|---|---|---|---|---|
| Ingredients | wt % | wt % | wt % | wt % | wt % | wt % | wt % |
| Cariprazine hydrochloride | 0.0 | 0.39 | 0.78 | 1.17 | 2.34 | 3.50 | 4.67 |
| Coprocessed mannitol and starch | 93.0 | 92.61 | 92.22 | 91.83 | 90.66 | 89.50 | 88.33 |
| Sodium Starch Glycolate | 5.0 | 5.0 | 5.0 | 5.0 | 5.0 | 5.0 | 5.0 |
| Malic acid | 1.0 | 1.0 | 1.0 | 1.0 | 1.0 | 1.0 | 1.0 |
| Sodium Stearyl Fumarate | 1.0 | 1.0 | 1.0 | 1.0 | 1.0 | 1.0 | 1.0 |
| Total (wt %) | 100 | 100 | 100 | 100 | 100 | 100 | 100 |

Results of the e-tongue measurements were evaluated using PLS (Partial Least Squares) regression, showing an estimation of the bitterness of each sample compared to quinine solutions (predicted “quinine bitterness”).

Table 3 and FIG. 2 illustrate the results of the above-described measurements showing that all the tested formulations are below the human bitterness taste threshold represented by Q2.

TABLE 3
Predicted quinine bitterness of different samples

| Code | Cariprazine HCl content (wt %) | Predicted quinine bitterness |
|---|---|---|
| A30 | 0.0 | −33.96 |
| A30 | 0.39 | −40.72 |
| A32 | 0.78 | −19.56 |
| A33 | 1.17 | 5.82 |
| A34 | 2.34 | 6.02 |
| A35 | 3.50 | 13.84 |
| A36 | 4.67 | 10.48 |

### EXAMPLE 3. DISINTEGRATION EFFICACY. FRIABILITY AND RESISTANCE TO CRUSHING TEST

The disintegration efficacy of the pharmaceutical tablets was measured in accordance with the European Pharmacopoeia (9^{th} Edition, 2.9.1. Disintegration of tablets and capsules Test A). This test is provided to determine whether tablets or capsules disintegrate within the prescribed time when placed in a liquid medium under the experimental conditions.

For the purposes of this test, disintegration does not imply the complete dissolution of the unit or even of its active constituents. Complete disintegration is defined as that state in which any residue of the unit is a soft mass having no palpably firm core (except fragments of insoluble coating or capsule shell remaining on the screen of the test apparatus or adhering to the lower surface of the discs, if used).

The friability and the resistance to crushing of the pharmaceutical tablets were measured in accordance with the European Pharmacopoeia (9^{th} Edition, 2.9.7. Friability of uncoated tablets; 2.9.8. Resistance to crushing of tablets).

TABLE 4
Qualitative and quantitative composition of different exemplary formulations

| | F1 | F2 | F3 | F4 | F5 | F6 | F7 | F8 | F9 | F10 |
|---|---|---|---|---|---|---|---|---|---|---|
| Ingredients | wt % | wt % | wt % | wt % | wt % | wt % | wt % | wt % | wt % | wt % |
| Cariprazine | 1.17 | 1.17 | 1.17 | 1.17 | 1.17 | 1.17 | 1.17 | 2.34 | 20.24 | 10.12 |
| HCl | | | | | | | | | | |
| Coprocessed | 97.83 | 95.83 | 93.83 | 89.83 | 87.83 | 85.83 | 83.83 | 86.66 | 68.76 | 78.88 |
| mannitol | | | | | | | | | | |
| and starch | | | | | | | | | | |
| Sodium | 0 | 2 | 4 | 8 | 10 | 12 | 14 | 5 | 5 | 5 |
| Starch | | | | | | | | | | |
| Glycolate | | | | | | | | | | |
| Sodium | 1 | 1 | 1 | 1 | 1 | 1 | 1 | 1 | 1 | 1 |
| Stearyl | | | | | | | | | | |
| Fumarate | | | | | | | | | | |
| Malic acid | — | — | — | — | — | — | — | 5 | 5 | 5 |
| Total (wt %) | 100 | 100 | 100 | 100 | 100 | 100 | 100 | 100 | 100 | 100 |

TABLE 5
Physical properties of the exemplary formulations of Table 4

| | F1 | F2 | F3 | F4 | F5 | F6 | F7 | F8 | F9 | F10 |
|---|---|---|---|---|---|---|---|---|---|---|
| Average hardness [N] | 67 | 65 | 61 | 62 | 62 | 49 | 56 | 53 | 50 | 52 |
| Disintegration [s] | 19 | 15 | 29 | 21 | 18 | 17 | 25 | 11 | 7 | 17 |
| Friability % | 0.60 | 0.60 | 0.40 | 1.10 | 0.50 | 0.90 | 1.00 | 0.39 | 0.31 | 0.31 |

TABLE 6
Qualitative and quantitative composition of different exemplary formulations

| | F11 | F12 | F13 | F14 | F15 | F16 | F17 | F18 |
|---|---|---|---|---|---|---|---|---|
| Ingredients | wt % | wt % | wt % | wt % | wt % | wt % | wt % | wt % |
| Cariprazine HCl | 1.17 | 1.17 | 1.17 | 1.17 | 1.17 | 1.17 | 1.17 | 1.17 |
| Coprocessed | 81.83 | 79.83 | 75.83 | 69.83 | 61.83 | 56.83 | 51.83 | 49.83 |
| mannitol and | | | | | | | | |
| starch | | | | | | | | |
| Sodium Starch | 16 | 18 | 22 | 28 | 36 | 41 | 46 | 48 |
| Glycolate | | | | | | | | |
| Sodium Stearyl | 1 | 1 | 1 | 1 | 1 | 1 | 1 | 1 |
| Fumarate | | | | | | | | |
| Total (wt %) | 100 | 100 | 100 | 100 | 100 | 100 | 100 | 100 |

TABLE 7
Physical properties of the exemplary formulations of Table 6

| | F11 | F12 | F13 | F14 | F15 | F16 | F17 | F18 |
|---|---|---|---|---|---|---|---|---|
| Average hardness [N] | 56 | 29 | 21 | nm | 23 | nm | 21 | nm |
| Disintegration [s] | 27 | 27 | 21 | 39 | 37 | 25 | 48 | 39 |
| Friability % | 0.40 | 4.12 | 10.63 | 18.34 | 2.70 | 91.96 | 1.10 | 2.25 |

(“nm”: not measurable)

Tables 4 to 7 show that concentrations of the disintegrant (sodium starch glycolate) below 15 wt % do not negatively affect the disintegration time of the pharmaceutical tablet while disintegrant concentrations above this value affect the friability in a substantial way. According to the literature, such low disintegrant concentrations would not allow an acceptable disintegration time of the tablet.

### EXAMPLE 4. DISSOLUTION TEST

ODT products usually are developed either for a more convenient form of administration or for more rapid onset of action of the drug, therefore they are basically immediate release products. Immediate release is defined by the EMA (EMA/CHMNP/CVMP/QWP/336031/2017) as a drug product where at least 75% (Q) of the active substance is dissolved within 45 minutes. A dissolution specification of a product should ensure batch to batch consistency and, ideally, signal potential problems with in vivo bioavailability. Therefore, reproducible dissolution behavior with a low standard deviation is necessary to ensure consistent quality during the shelf-life and also the lifetime of a product. The dissolution behavior of the different dose strengths of the same product should be similar, especially if the dissolution properties of the different dose strengths of its other marketed dosage forms are similar.

During the formulation experiments it was found, that despite the rapid tablet disintegration, the release of cariprazine was not adequate at higher dose strengths, i.e., the average dissolution was not close to complete, and the standard deviation of the dissolution values measured at 15 minutes was higher compared to the lower dose strengths.

The pharmacokinetic parameters and the therapeutic effect of a drug could be subject to uncertainty if the dissolution release kinetics are not the same for all of the dose strengths. Slowing of dissolution can change the onset of drug action and unevenness in dissolution can result in pharmacokinetic variability.

It was found that disintegrants could neither increase remarkably the in vitro disintegration of the tablets nor the dissolution of cariprazine from the tablets. Increasing the amount of the super disintegrant resulted in a poorer powder quality in terms of flow properties and produced more friable tablets without significantly improving the dissolution of cariprazine.

Several different excipients were tested to improve the dissolution of cariprazine, but most of them were not suitable for palatable orally disintegrating tablets. During the formulation experiments glidants were tested to improve the powder and tablet properties, and it was surprisingly found that the use of colloidal silicon dioxide improves the dissolution of cariprazine without interfering with other parameters of the tablet.

The dissolution of tablets comprising 1.5 mg of cariprazine without colloidal silicon dioxide was almost complete after 15 minutes and each measured tablet showed similar dissolution, i.e., the min-max range of the measurements was low (Table 8 and 9). On the other hand, the dissolution of the tablets comprising 6 mg of cariprazine without colloidal silicon dioxide was lower at 15 minutes, and the individual measurement points showed a higher standard deviation, i.e., the min-max range of the measurements was high (Table 8 and 9). In the clinical practice this may cause changes in pharmacokinetic parameters when switching to a higher dose strength and may increase the variability of blood levels of the drug.

However, even smaller amounts of colloidal silicon dioxide significantly improved the dissolution of cariprazine at higher dose strengths and it became more similar to the dissolution of lower dose strengths. Both the average dissolution and the standard deviation of the dissolution were improved (Table 8 and 9).

TABLE 8
Qualitative and quantitative composition of different exemplary formulations

| | F19 | F20 | F21 | F22 | F23 | F24 | F25 |
|---|---|---|---|---|---|---|---|
| Ingredients | wt % | wt % | wt % | wt % | wt % | wt % | wt % |
| Dose strength | 1.5 mg | 1.5 mg | 6 mg | 6 mg | 6 mg | 6 mg | 6 mg |
| Cariprazine HCl | 1.17 | 1.17 | 4.67 | 4.67 | 4.67 | 4.67 | 4.67 |
| Coprocessed | 91.33 | 91.33 | 87.83 | 87.83 | 87.08 | 87.08 | 87.33 |
| mannitol and starch | | | | | | | |
| Sodium starch | 5 | 5 | 5 | 5 | 5 | 5 | 5 |
| glycolate | | | | | | | |
| Malic acid | 1 | 1 | 1 | 1 | 1 | 1 | 1 |
| Hydrated colloidal | | | | | | 0.50 | |
| silicon dioxide | | | | | | | |
| Anhydrous colloidal | | | | | 0.50 | | 0.25 |
| silicon dioxide | | | | | | | |
| Sodium stearyl | 1.50 | 1.50 | 1.50 | 1.50 | 1.75 | 1.75 | 1.75 |
| fumarate | | | | | | | |
| Total (wt %) | 100 | 100 | 100 | 100 | 100 | 100 | 100 |

TABLE 9
Dissolution parameters of different exemplary formulations

| Dissolution values (n = 6) | F19 | F20 | F21 | F22 | F23 | F24 | F25 |
|---|---|---|---|---|---|---|---|
| Average dissolution at 15 min | 97 | 95 | 90 | 86 | 90 | 102 | 95 |
| lowest value at 15 min | 95 | 93 | 82 | 79 | 87 | 99 | 92 |
| highest value at 15 min | 101 | 97 | 97 | 92 | 93 | 106 | 97 |
| undissoluted amount at 15 min | 3 | 3 | 10 | 9 | 5 | 3 | 4 |
| min-max range at 15 min | 6 | 4 | 15 | 13 | 6 | 7 | 5 |
| Passing an immediate release | yes | yes | no | no | yes | yes | yes |
| dissolution specification | | | | | | | |
| (Q = 80% @ 15 min) at the level | | | | | | | |
| of S1* | | | | | | | |

(*level of acceptance criteria for dissolution measured from 6 tablets according to Ph. Eur. 2.9.3.)

The tablets according to the present invention show similar dissolution kinetics for all dose strengths with low variability, thus meeting therapeutic and safety requirements. Consequently, the overall quality of the product is improved.

### EXAMPLE 5. STABILITY TEST

The stability of the pharmaceutical tablets was measured in accordance with the CPMP/ICH/2736/99 (Q1A(R2) Stability Testing of New Drug Substances and Products (EC, Europe—Adopted by CPMP, March 2003).

Samples were packed in Cold—Alu form blister (samples “A”) and PVC/PE/PVDC-Alu packaging material (samples “B”) and they were stored at accelerated condition (40° C./70% RH) and long-term conditions (30° C./65% RH and 25° C./60% RH) as well. The tablets according to the present invention show a good stability over time (up to 6 months), even under high temperature and humidity conditions.

1. An orally disintegrating pharmaceutical tablet comprising:
between 0.1 and 21 wt % of cariprazine or a pharmaceutically acceptable salt thereof,
between 0.1 and 15 wt % of a disintegrant,
between 0.1 and 5 wt % of colloidal silicon dioxide,
between 0.1 and 10 wt % of a taste masking agent,
between 0.1 and 5 wt % of a lubricant, and
up to 100 wt % of a diluent,
the percentage being expressed with regard to the total weight of the pharmaceutical tablet.

2. The pharmaceutical tablet according to claim 1, wherein the pharmaceutically acceptable salt of cariprazine is a hydrochloride salt.

3. The pharmaceutical tablet according to claim 1, wherein the colloidal silicon dioxide is anhydrous colloidal silicon dioxide or hydrated colloidal silicon dioxide.

4. The pharmaceutical tablet according to claim 3, wherein the colloidal silicon dioxide is hydrated colloidal silicon dioxide.

5. The pharmaceutical tablet according to claim 1, wherein the taste masking agent is an organic acid.

6. The pharmaceutical tablet according to claim 5, wherein the organic acid is selected from the group consisting of: oxalic acid, maleic acid, succinic acid, citric acid, tartaric acid, malic acid, and combinations thereof.

7. The pharmaceutical tablet according to claim 1, wherein the disintegrant is selected from the group consisting of: sodium starch glycolate, crospovidone, croscarmellose sodium, and combinations thereof.

8. The pharmaceutical tablet according to claim 1, wherein the diluent comprises mannitol, dextrates, isomalt, sorbitol, or mixtures thereof; and starch.

9. The pharmaceutical tablet according to claim 8, wherein the diluent is a mixture of 78.0-82.0 wt % D-mannitol and 15.0-19.0 wt % maize starch, the percentage being expressed with regard to the total weight of the diluent.

10. The pharmaceutical tablet according to claim 1, wherein the lubricant is magnesium stearate or sodium stearyl fumarate.

11. The pharmaceutical tablet according to claim 1, wherein the disintegrant is present in an amount between 0.1 wt % and 10 wt %.

12. The pharmaceutical tablet according to claim 1, wherein the colloidal silicon dioxide is present in an amount between 0.1 wt % and 2 wt %.

13. The pharmaceutical tablet according to claim 1, wherein the taste masking agent is present in an amount between 0.1 and 5 wt %.

14.-17. (canceled)

18. A method of treating a pathological condition which requires the modulation of dopamine receptors in a patient in need of treatment thereof, wherein the pathological condition is selected from the group consisting of: psychoses, drug abuse, cognitive impairment accompanying schizophrenia, mild-to-moderate cognitive deficits, dementia, psychotic states associated with dementia, eating disorders, attention deficit disorders, hyperactivity disorders in children, psychotic depression, bipolar disorder, paranoid and delusional disorders, dyskinetic disorders, anxiety, sexual dysfunction, sleep disorders, emesis, aggression and autism, wherein the method comprises administering the pharmaceutical tablet according to claim 1 to the patient.

19. The method according to claim 18, wherein the pathological disorder is schizophrenia, bipolar disorder, or autism.
